(19) United States
(12) Patent Application Publication
(10) Pub. No.: US 2022/0240826 A1
(43) Pub. Date: Aug. 4, 2022

(54) ELECTRONIC FUNCTIONAL MEMBER, METHOD FOR MANUFACTURING SAME, AND BIOLOGICAL MEASUREMENT SENSOR

(71) Applicant: The University of Tokyo, Bunkyo-Ku, Tokyo (JP)

(72) Inventors: Takao Someya, Bunkyo-ku Tokyo (JP); Akihito Miyamoto, Bunkyo-ku, Tokyo (JP); Yan Wang, Bunkyo-ku, Tokyo (JP); Sunghoon Lee, Bunkyo-ku, Tokyo (JP); Shiho Nagai, Bunkyo-ku, Tokyo (JP); Ikue Kawashima, Bunkyo-ku, Tokyo (JP)

(21) Appl. No.: 17/436,251

(22) PCT Filed: Mar. 6, 2020
(86) PCT No.: PCT/JP2020/009645

§ 371 Date: Apr. 18, 2022

(30) Foreign Application Priority Data
Mar. 7, 2019 (JP) 2019-041623

(51) Int. Cl. A61B 5/263; D04H 1/4309; D04H 1/728

(52) U.S. Cl. CPC A61B 5/263; A61B 2562/125; D04H 1/728; D04H 1/4309

(57) ABSTRACT

[Problem] Provided is an electronic functional member of superior wear resistance. [Solution] The present invention comprises a fiber network constituted by a water-soluble resin and a poorly water-soluble resin, and an electroconductive member formed on the fiber network. The water-soluble resin and poorly water-soluble resin are, for example, polyvinyl alcohol derivatives. In accordance with an embodiment of the electronic functional member according to the present invention, the fiber network is formed by layering a first fiber network constituted by fibers containing a water-soluble first resin and a second fiber network constituted by fibers containing a poorly water-soluble second resin. Alternatively, the fiber network may be constituted by fibers containing the water-soluble first resin and fibers containing the poorly water-soluble second resin. Alternatively, the fiber network may be constituted by fibers containing the water-soluble first resin and the poorly water-soluble second resin.

### CROSS-REFERENCE TO RELATED APPLICATIONS

This application is the national stage application of International Patent Application No. PCT/JP2020/009645 filed on Mar. 6, 2020 and entitled “ELECTRONIC FUNCTIONAL MEMBER, METHOD FOR MANUFACTURING SAME, AND BIOLOGICAL MEASUREMENT SENSOR”, which claims priority to Japanese Patent Application No. 2019-041623 filed on Mar. 7, 2019 and entitled “ELECTRONIC FUNCTIONAL MEMBER, METHOD FOR MANUFACTURING SAME, AND BIOLOGICAL MEASUREMENT SENSOR” the entire contents of which are hereby incorporated by reference herein in their entireties.

### TECHNICAL FIELD

This invention relates to an electronic functional member and a method for manufacturing the same, and further, a biometric sensor configured using the electronic functional member.

### BACKGROUND

Recently, flexible electronics has attracted a lot of attention because softness of the materials provides various applications. Particularly, in accordance with the worldwide aging of the society, the health care field has been drawing increasing attention. For example, the flexible electronics has been attracting attention as means for directly obtaining biological information from cells and tissues by attaching to the surface of the human body and inside the body.

Generally, while the flexible electronics is manufactured by forming an electronics device on a flexible substrate, its flexibility cannot be said to be sufficient. Therefore, the surface followability cannot be said to be sufficient, and thus it is impossible to obtain information with high accuracy and to sufficiently reduce the uncomfortable feeling and the like during the attachment.

To solve such a problem, there has been proposed an electronic functional member in which a fiber network of a nanofiber made of water-soluble polyvinyl alcohol (PVA) is formed by an electrospinning method, and gold is vapor-deposited thereon to form an electrode layer, thereby providing sufficiently high surface followability, stretchability in a lateral direction, permeabilities of gas and water, and transparency (for example, Non-patent Document 1).

Non-patent Document 1: Akihito Miyamoto et.al., Nature Nanotechnology 12, 907 (2017)

However, when the electronic functional member of the above-described prior art is directly attached to the skin to be used, there is a room for improving the durability because of breaking of the electrode layer due to rubbing, easily peeling off from the skin in a case of wetting with water, and the like.

Therefore, the inventors involved in this application have been dedicated to the examination and found that the durability is improved with a fiber network configured to include two types of resins mutually different in solubility to water.

This invention is made in consideration of the above-described point, and an object of this invention is to provide an electronic functional member improved in durability.

### SUMMARY

To achieve the above-described object, an electronic functional member of the invention includes a fiber network and a conductive member. The fiber network is configured to include a resin. The fiber network is partially solved in water and partially remains when immersed in water. The conductive member is formed on the fiber network. Here, the resin is, for example, a polyvinyl alcohol derivative.

According to an embodiment of the electronic functional member of the invention, the fiber network includes a first resin and a second resin as the resin, and the first resin and the second resin are mutually different in solubility to water. In a preferred embodiment, the fiber network is configured by stacking a first fiber network including a fiber containing the first resin and a second fiber network including a fiber containing the second resin. In another preferred embodiment, the fiber network includes a fiber containing the first resin and a fiber containing the second resin. In further another preferred embodiment, the fiber network includes a fiber containing the first resin and the second resin.

In the electronic functional member of the invention, an occupancy of the fiber in the fiber network is preferably 20% to 90%, and more preferably the occupancy is 30% to 70%.

According to a method for manufacturing an electronic functional member of the invention, the method includes: a step of forming a first fiber network that includes a fiber containing a water-soluble first resin; a step of forming a second fiber network that includes a fiber containing a second resin that is water-soluble and changes to being poorly water-soluble after heating and pressure bonding; a step of changing the second resin to being poorly water-soluble by heating and pressure bonding the second fiber network; and a step of stacking the first fiber network, the second fiber network, and a conductive member.

According to another embodiment of the method for manufacturing an electronic functional member of the invention, the method includes: a step of forming a fiber network that includes a fiber containing a first resin and a fiber containing a second resin, the first resin being water-soluble and remaining water-soluble after heating and pressure bonding, and the second resin being water-soluble and changing to being poorly water-soluble after the heating and the pressure bonding; a step of heating and pressure bonding the fiber network; and a step of disposing a conductive member on the fiber network.

According to another embodiment of the method for manufacturing an electronic functional member of the invention, the method includes: a step of forming a fiber network that includes a fiber containing a first resin and a second resin, the first resin being water-soluble and remaining water-soluble after heating and pressure bonding, and the second resin being water-soluble and changing to being poorly water-soluble after the heating and the pressure bonding; and a step of heating and pressure bonding the fiber network and further disposing a conductive member on the fiber network.

According to the electronic functional member of this invention, with the configuration including the resin solved in water and the resin that remains when the fiber network is immersed in water, the durability is improved compared with the conventional electronic functional member in which the fiber network of the nanofiber made of polyvinyl alcohol (PVA) is formed and gold is vapor-deposited thereon to form the electrode layer.

### BRIEF DESCRIPTION OF THE DRAWINGS

FIG. 1 includes schematic diagrams for describing a first electronic functional member.

FIG. 2 is a schematic diagram for describing a method for manufacturing the first electronic functional member.

FIG. 3 is a diagram illustrating a result of a water solubility test of resins.

FIG. 4 is a diagram indicating a difference of crystallinity depending on a heating temperature.

FIG. 5 includes schematic diagrams for describing a second electronic functional member.

FIG. 6 is a schematic diagram for describing a method for manufacturing the second electronic functional member.

FIG. 7 includes schematic diagrams for describing a third electronic functional member.

FIG. 8 is a schematic diagram for describing a method for manufacturing the third electronic functional member.

FIG. 9 includes diagrams illustrating results of an adhesion evaluation.

FIG. 10 includes photographs illustrating results of the adhesion evaluation.

FIG. 11 includes schematic diagrams for describing a fourth electronic functional member.

FIG. 12 includes schematic diagrams for describing a fifth electronic functional member.

FIG. 13 includes diagrams illustrating results of a water resistance test.

FIG. 14 is a schematic diagram for describing a biometric sensor.

### DETAILED DESCRIPTION

While the following describes embodiments of the invention with reference to the drawings, shapes, sizes, and positional relationships of respective components are only schematically illustrated for understanding the invention. While the following describes preferred exemplary configurations of the invention, materials, numerical conditions, and the like of respective components are merely preferable examples. Therefore, the invention is not limited to the following embodiments, and many changes or modifications that can achieve the effects of the invention can be made without departing from the scope of the configuration of the invention.

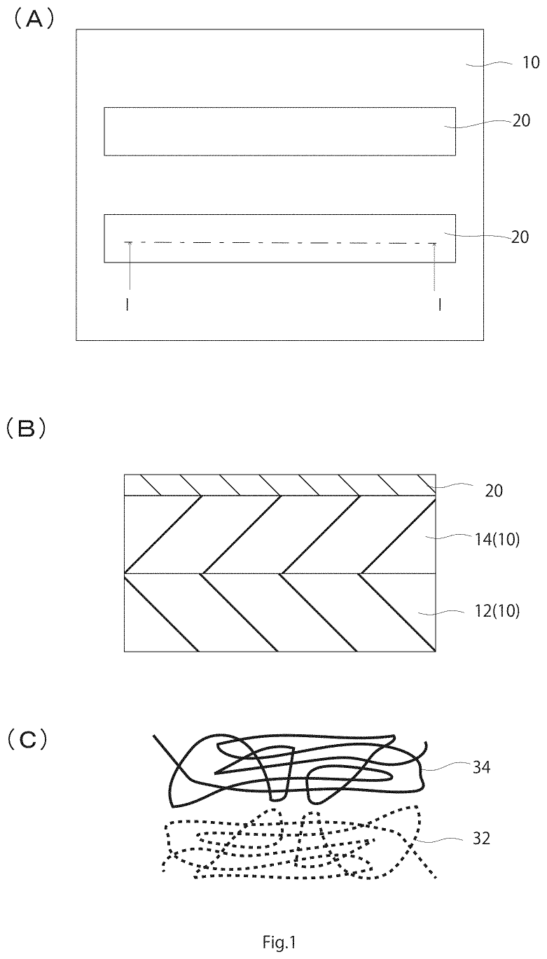

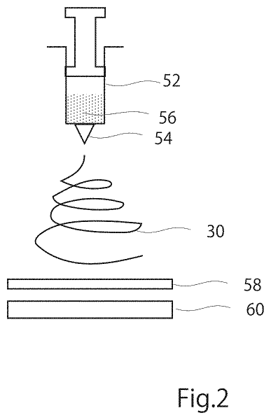

(Configuration and Manufacturing Method for First Electronic functional member) With reference to FIG. 1 and FIG. 2, an electronic functional member according to a first embodiment of the invention (hereinafter referred to as a first electronic functional member) will be described. FIG. 1 includes schematic diagrams for describing the first electronic functional member. FIG. 1(A) is a schematic plan view, FIG. 1(B) is a schematic cross-sectional view taken along a line I-I of FIG. 1(A), and FIG. 1(C) is a schematic diagram illustrating an outline of a fiber network 10 included in the first electronic functional member. FIG. 2 is a schematic diagram for describing a method for manufacturing the first electronic functional member.

The first electronic functional member is configured to include the fiber network 10 containing a resin and a conductive member 20 formed on the fiber network 10. The fiber network 10 includes a first fiber network 12 containing a first resin and a second fiber network 14 containing a second resin, which are stacked. Here, the first resin and the second resin are mutually different in solubility to water. The conductive member 20 is disposed on the second fiber network 14 of the fiber network 10.

While the first fiber network 12 and the second fiber network 14 are formed by any appropriate conventionally-known method, for example, the first fiber network 12 and the second fiber network 14 are formed by injecting a resin composition by an electrospinning method (FIG. 2).

In the electrospinning method, a solution 56 in a syringe 52 is extruded while applying a high voltage between a needle 54 of the syringe 52 and a conductive sheet 60. At this time, by an electric potential difference between the needle 54 and the conductive sheet 60, the solution 56 is rapidly extracted from the syringe 52, and sprayed toward the conductive sheet 60. By sandwiching a support body 58 between the syringe 52 and the conductive sheet 60, the solution 56 is sprayed on the support body 58 to form a resin composition 30, thus producing a fiber network. The solution 56 is one in which the resin composition 30 is dissolved in a solvent, and the solvent is mostly vaporized between the needle 54 and the support body 58. Thus, the first fiber network 12 and the second fiber network 14 are each formed. For the support body 58, the material and the like are not specifically limited insofar as the support body 58 functions to support the fiber network 10.

When the fiber in the fiber network 10 is a flexible material like silicone and polyurethane, the fiber is easily stretched to be broken. In a case of a hard material like polycarbonate, the followability to the skin becomes poor. In view of this, the fiber in the fiber network is preferably a fiber with a Young's modulus in a range of 500 MPa to 8000 MPa, and more preferably a fiber in a range of 1000 MPa to 5000 MPa.

As the resin constituting the fiber in the fiber network 10, for example, a polyvinyl alcohol (PVA) derivative with the Young's modulus of about 2000 to 4000 MPa is usable.

The first fiber network 12 includes a first resin 32 solved in water when immersed in water. The first resin 32 is, for example, a water-soluble resin having a basic structure indicated by a formula (1) below. The first fiber network 12 includes a fiber containing the first resin 32.

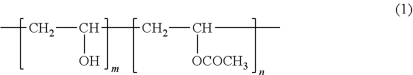

Here, the first resin 32 only needs to be selected from a saponification degree of 75 or more [mol %] and a viscosity of 5.0 to 65.0 [mm²/s], and a PVA derivative of the saponification degree of 86.5 to 89.0 [mol %] and the viscosity of 15.3 to 55.7 [mm²/s] is preferably used. The saponification degree is obtained by {m/(m+n)}×100 [mol %]. The viscosity is measured under a condition of 4% of a water solution at 20° C. according to Japanese Pharmaceutical Excipients (JPE).

As the PVA derivative having the above-described properties, EG-18P, 22P, 30P, 40P, and 48P of GOHSENOL EG series by Mitsubishi Chemical Corporation are available.

The second fiber network 14 is configured to include a poorly water-soluble second resin 34 that is not solved in water and remains when immersed in water. The second resin 34 having the poor water solubility is obtained by heating and pressure-bonding the first resin 32 having the water solubility. For example, the second resin 34 is obtained by a thermocompression bonding of a PVA derivative that has the basic structure indicated by the above-described formula (1), the saponification degree of 86.5 to 89.0 [mol %], and the viscosity of 15.3 to 34.5 [mm²/s]. As this PVA derivative, the above-described EG-18P, 22P, and 30P are used. While the condition of the thermocompression bonding can be appropriately set as necessary, for example, a condition in which the temperature is in a range of 90 to 180° C. and a press is performed for 1 to 5 minutes can be employed.

When the thermocompression bonding is performed to the resin of the PVA derivative, changing the heating temperature and the heating period varies the solubility to water. For example, a fiber containing a resin subjected to the thermocompression bonding at 180° C. for three minutes is less likely to be solved in water compared with a fiber subjected to the thermocompression bonding at 120° C. for one minute. Here, the point that a water-soluble resin changes to being poorly water-soluble is described in Non-patent Document 2 (Polymer Vol. 39, No. 18 pp. 4295-4302, 1998).

Note that, while the poorly water-soluble resin is obtained using a thermocompression bonding apparatus here, the heating method is not limited thereto, and various kinds of apparatuses and methods are usable. The heating and the pressure bonding may be individually performed instead of simultaneously performing the heating and the pressure bonding.

The first fiber network 12 and the second fiber network 14 are stacked and bonded by any appropriate means, thereby obtaining the fiber network 10.

The fiber network 10 includes the first resin and the second resin mutually different in solubility to water as the resins. The first resin 32 is solved in water when immersed in water. Meanwhile, the second resin 34 is not immediately solved when immersed in water.

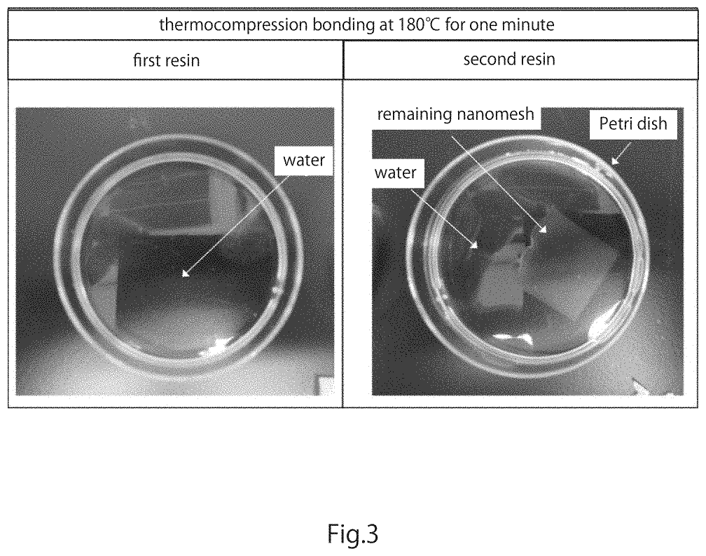

FIG. 3 illustrates a result of examining the water solubility when the resin is immersed in water. Here, EG-48P as the first resin and EG-22P as the second resin are both subjected to the thermocompression bonding at 180° C. for one minute. As illustrated in FIG. 3, it is seen that the first resin is entirely solved in water, and the second resin partially remains. Thus, it is obvious that the solubility is different between the first resin and the second resin through the thermocompression bonding.

Therefore, when the first electronic functional member is immersed in water for a predetermined period, while the first resin 32 constituting the first fiber network 12 is immediately almost entirely solved in water, the second resin 34 remains by a predetermined amount. The inventors involved in this application has found that this action improves the wear resistance as described below.

While the first fiber network 12 and the second fiber network 14, which is not subjected to the thermocompression bonding, are in an amorphous state, the second fiber network 14 is crystallized when the second fiber network 14 is subjected to the thermocompression bonding. Therefore, when a structural analysis by an X-ray diffraction is performed, for example, an X-ray diffraction angle, a half-value width, and an intensity vary depending on the crystallinity. Thus, the water-soluble resin and the poorly water-soluble resin can be discriminated also by the diffraction profile obtained from the X-ray diffraction.

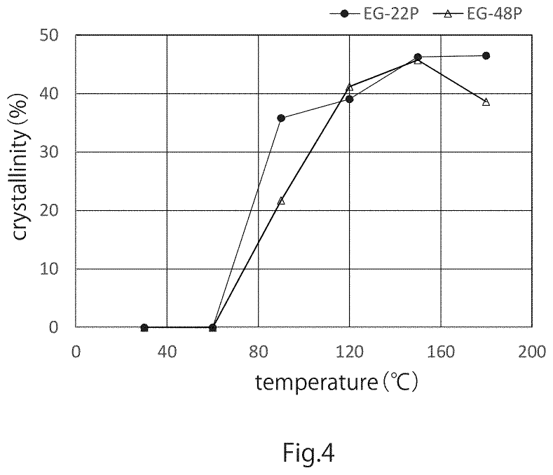

FIG. 4 illustrates a result of examining the difference in crystallinity between EG-22P and EG-48P depending on the heating temperature. In FIG. 4, the horizontal axis indicates the temperature (° C.) and the vertical axis indicates the crystallinity (%). The crystallinity is obtained from the structural analysis by the X-ray diffraction. For both of EG-22P and EG-48P, the crystallinity increases with the heating at the temperature of 90° C. or more. Especially, at the temperatures about 90° C. and 180° C., the crystallinity is different between EG-22P and EG-48P, and the crystallinity of EG-22P is higher at both temperatures. Accordingly, for example, when EG-48P is used as the first resin 32 and EG-22P is used as the second resin 34, by the heating at 180° C., the first resin 32 containing EG-48P with the low crystallinity indicates the water solubility, and the second resin 34 containing EG-22P with the high crystallinity indicates the poor water solubility.

Also for the resin components dissolved when the water-soluble resin and the poorly water-soluble resin are immersed in water, since the molecular weight of the resin can be easily measured by a common method for analyzing a high-polymer material, such as a gel permeation chromatography (GPC) method, the respective fibers can be discriminated.

The conductive member 20 can be formed using, for example, an evaporation method, a sputtering method, a chemical vapor deposition method, an inkjet method, a screen-printing method, a gravure printing method, and a flexography method. While FIG. 1(A) illustrates an exemplary configuration of two strip shapes as the electrodes including the conductive members 20, an appropriate shape may be employed depending on the usage.

The shape of the electrode including the conductive member 20 can be changed by performing a patterning as necessary. As the patterning method, film formation via a mask is the easiest and preferred.

The material constituting the conductive member 20 only needs to have a conductive property. For example, a metal such as copper, gold, aluminum, silver, and zinc is usable. From the aspect of the conductive property, particularly, copper and silver are preferred. In a case of using for a living body and the like, for suppressing unwanted reactions, the use of stable gold is preferred.

Here, when it is assumed that the electrode including the conductive member has a length L, a width w, a thickness d, a resistivity p, a resistance value R, and a fiber occupancy C in a fiber network, a network rate a defined by a formula (2) below is preferably 0.05 or more, and more preferably 0.1 or more.

α=πρL/2wdRC (2)

When the network rate a is small, positions at which the vertically intersecting fibers are mutually fused decrease. Consequently, it is considered that when a force, such as rubbing, is applied in a direction perpendicular to the thickness direction of the electrode, the vertically intersecting fibers are shifted to easily cause a wire disconnection. The network rate can be obtained by the formula (2) described above. With a scanning probe microscope, it can be confirmed whether the conductive members of the vertically intersecting fibers are electrically conducted or not.

The fiber occupancy C in the fiber network 10 is preferably 20% to 90%, and the occupancy C is further preferably 30% to 70%. The fiber network 10 includes a portion at which the nanofiber is formed and a portion provided with a void. Therefore, the “fiber occupancy C in the fiber network” means a proportion of the portion at which the nanofiber is formed in plan view. This occupancy can be obtained by taking photographs of the surface of the fiber network with a size of 1 mm×1 mm at any 10 points, subsequently obtaining areas of portions at which the nanofiber is formed in the respective photographs, and obtaining an average value of them. When the fiber occupancy C in the fiber network 10 is excessively high, the permeabilities of gas and water content decrease. Meanwhile, since the occupancy of the conductive member 20 has a positive correlation with the fiber occupancy C in the fiber network 10, when the fiber occupancy C in the fiber network 10 is excessively low, the conductive member 20 becomes sparse, thus making it difficult to ensure the sufficient conductive property.

The fiber constituting the fiber network 10 preferably has a diameter of 100 nm to 10 μm. Particularly, for example, in a case of using by attaching to a skin, 200 nm to 2 μm, what is called a nanofiber is preferred. In this range, an electronic functional member having a sufficient strength and high permeabilities of gas and water content can be obtained. The diameter of the nanofiber can be obtained by, for example, measuring cross-sectional surfaces of the resin composition at any 10 points with the scanning electron microscope and obtaining an average value of diameters of them.

Here, a fiber network in which a fiber constituting the fiber network is a nanofiber is referred to as a nanomesh, and an electronic functional member in which a fiber network is a nanomesh is referred to as a nanomesh electrode in some cases.

(Use Method and Properties of First Electronic functional member) The electronic functional member is placed on an object to be attached, such as a skin, such that the first fiber network 12 contacts the object to be attached, and water is provided. Thus, mainly the first fiber network 12 is dissolved, and consequently, the electronic functional member is attached to the skin.

In a state of being attached to an artificial skin as the object to be attached, the conductive member 20 of the electronic functional member was repeatedly rubbed from the front surface side, and the number of time until the wire disconnection was measured. This measuring method will be described.

First, an EVA (Ethylene-Vinyl Acetate) sponge sheet is fixed with an adhesive double coated tape in a shape of a glass slide having a thickness of 1 mm. A substrate body in which an artificial skin (manufactured by Beaulax) having a thickness of 100 μm is attached to the EVA sponge sheet is prepared. Subsequently, nanomesh electrodes of 4 mm×30 mm are attached to the substrate body with water, thus producing an evaluation sample. Here, the three nanomesh electrodes were formed at intervals of 2.5 mm

Next, a friction and wear test is conducted using a friction and wear tester (FPR2200 manufactured by RHESCA CO., LTD). The friction and wear test is a testing method in which a measurement indenter is pressed with a constant weight and slid. Here, a polyurethane ball of 5 mm was used as the measurement indenter, and the weight load was 50 g. The evaluation sample was fixed to a linearly reciprocating slide unit of the friction and wear tester and slid at 20 mm/second, thus measuring the number of times until the wire disconnection of the nanomesh electrodes.

The determination whether the wire was disconnected or not was made by measuring electrical resistances in a longitudinal direction of the nanomesh electrodes using a commercially available tester, and the resistance value of 1000 Ω or more was determined as the wire disconnection.

The measurement result is indicated in a table below. The table indicates Examples of the first electronic functional member and Comparative Examples of conventional electronic functional members. Here, the examples in which PVA derivatives having the basic structures indicated by the above-described formula (1), the saponification degrees of 86.5 to 89.0 [mol %], and the different viscosities were used as the resins constituting the fiber networks are indicated. The resins constituting the respective fiber networks are discriminated by the viscosity [mm²/s].

TABLE 1

| | | | the number |
|---|---|---|---|
| | fiber networks (viscosity [mm²/s]) | | of times until |
| | the first | the second | the wire |
| | fiber networks | fiber networks | disconnection |
| example 1 | 19.0-25.6 | 25.5-34.5 | >1000 |
| | | thermocompression bonding | |
| | | (180° C. 5 min.) | |
| example 2 | 19.0-25.6 | 25.5-34.5 | >1000 |
| | | thermocompression bonding | |
| | | (180° C. 3 min.) | |
| example 3 | 19.0-25.6 | 25.5-34.5 | >1000 |
| | | thermocompression bonding | |
| | | (180° C. 1 min.) | |
| example 4 | 41.3-55.7 | 19.0-25.6 | >1000 |
| | | thermocompression bonding | |
| | | (180° C. 5 min.) | |
| example 5 | 41.3-55.7 | 19.0-25.6 | >1000 |
| | | thermocompression bonding | |
| | | (180° C. 3 min.) | |
| example 6 | 41.3-55.7 | 19.0-25.6 | >1000 |
| | | thermocompression bonding | |
| | | (180° C. 1 min.) | |
| example 7 | 15.3-20.7 | 19.0-25.6 | >1000 |
| | | thermocompression bonding | |
| | | (180° C. 5 min.) | |
| example 8 | 25.5-34.5 | 19.0-25.6 | >1000 |
| | | thermocompression bonding | |
| | | (180° C. 5 min.) | |
| example 9 | 36.6-49.4 | 19.0-25.6 | >1000 |
| | | thermocompression bonding | |
| | | (180° C. 5 min.) | |

| | | |
|---|---|---|
| comparative | 15.3-20.7 no thermocompression bonding | 100 |
| example 1 | | |
| comparative | 19.0-25.6 no thermocompression bonding | 300 |
| example 2 | | |
| comparative | 25.5-34.5 no thermocompression bonding | 50 |
| example 3 | | |
| comparative | 41.3-55.7 no thermocompression bonding | 50 |
| example 4 | | |
| comparative | 41.3-55.7 no thermocompression bonding | 50 |
| example 5 | | |
| comparative | 15.3-20.7 thermocompression bonding | 100 |
| example 6 | (180° C. 5 min.) | |
| comparative | 19.0-25.6 thermocompression bonding | 200 |
| example 7 | (180° C. 5 min.) | |

The thermocompression bonding of the second fiber network in Examples were performed at 180° C. for 1 to 5 minutes, and in the thermocompression bonding in Comparative Examples, the press was performed at 180° C. for 5 minutes.

In each of Example, the number of times until the wire disconnection is 1000 times or more. Note that, here, since 1000 times is the end even when the wire is not disconnected, 1000 times means 1000 times or more. In contrast, in Comparative Examples of the conventional configurations, the number of times until the wire disconnection is 50 to 300 times.

Here, Comparative Examples 1 to 5 are cases where the fiber network includes only the water-soluble resin, and Comparative Examples 6 and 7 are cases where the fiber network includes only the poorly water-soluble resin.

Thus, it is seen that when the fiber network includes both of the water-soluble resin and the poorly water-soluble resin, the wear resistance is improved compared with any of the case where the fiber network includes only the water-soluble resin and the case where the fiber network includes only the poorly water-soluble resin.

The fiber network of the present invention can be attached without easily damaging the electrode even when attached to the skin with water, and the resistance against water was also improved compared with the conventional products (for example, Comparative Examples 1 to 5).

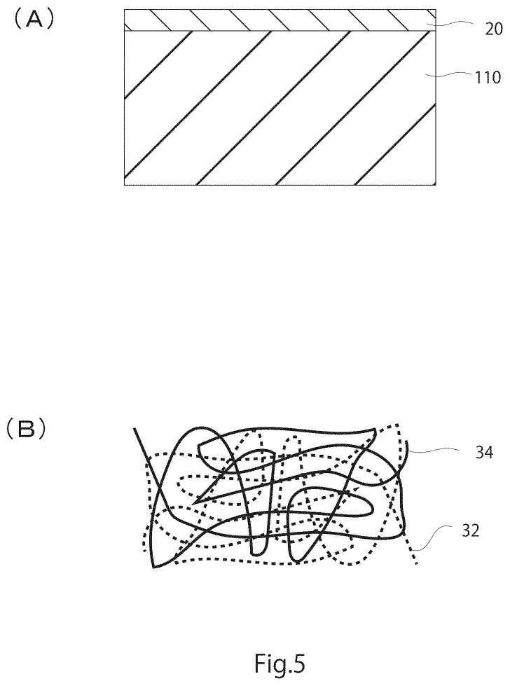

(Second Electronic functional member) With reference to FIG. 5, an electronic functional member according to a second embodiment of the invention (hereinafter referred to as a second electronic functional member) will be described. FIG. 5(A) is a schematic diagram for describing the second electronic functional member, and FIG. 5(B) is a schematic diagram illustrating an outline of a fiber network included in the second electronic functional member. FIG. 5(A) is a schematic cross-sectional view taken along a line similar to the line I-I of FIG. 1(A).

In the second electronic functional member, a fiber network 110 is configured to include the first resin and the second resin which are water-soluble and different in solubility to water after the thermocompression bonding. The first resin 32 is, for example, EG-40P or EG-48P, and the second resin 34 is EG-18P to 30P.

When the thermocompression bonding is performed to the fiber network 110 that includes the fiber containing the first resin 32 and the fiber containing the second resin 34, the solubility to water of the second resin 34 changes, and the second resin 34 becomes to be less likely to be solved in water compared with the first resin 32.

For example, when EG-48P is used as the first resin 32 and EG-22P is used as the second resin 34, through the thermocompression bonding, while the first resin 32 indicates the water solubility, the second resin 34 indicates the poor water solubility.

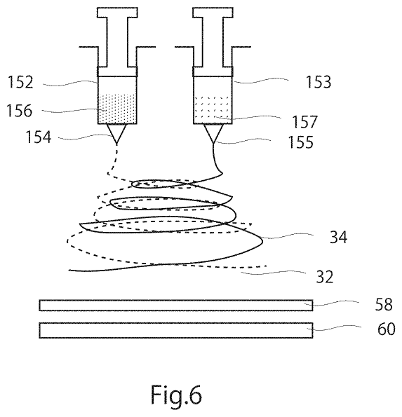

With reference to FIG. 6, a method for manufacturing the second electronic functional member will be described. The second electronic functional member is manufactured by an electrospinning method using two nozzles.

A first solution 156 in a first syringe 152 is sprayed to the support body 58 from a first needle 154. Meanwhile, a second solution 157 in a second syringe 153 is sprayed to the support body 58 from a second needle 155. The first solution 156 is one in which the first resin 32 is dissolved in a solvent. The second solution 157 is one in which the second resin 34 is dissolved in a solvent. The fiber containing the first resin 32 is formed through the first needle 154, and the fiber containing the second resin 34 is formed through the second needle 155.

Because of the similarity to the method for manufacturing the first electronic functional member except that the solutions in which the mutually different resins are dissolved are filled in the two syringes and the solutions are simultaneously sprayed from the two needles, the description will be omitted.

Consequently, by performing heating and pressure bonding of the fiber network 110 that includes the fiber containing the first resin 32 that is water-soluble and remains water-soluble after the heating and the pressure bonding and the fiber containing the second resin 34 that is water-soluble and changes to being poorly water-soluble after the thermocompression bonding, the second electronic functional member in which the fiber network 110 includes the first resin 32 and the second resin 34 mutually different in solubility to water is obtained.

In the second electronic functional member in which the fiber network 110 includes the fiber containing the first resin 32 and the fiber containing the second resin 34 in mixture, easily damaging the electrode with water during attaching to the skin was allowed to be suppressed. Furthermore, the resistance against water was significantly improved without peeling off regardless of attaching to the artificial skin and immersing in water for five minutes, and the wire was not disconnected until 500 times in the wear resistance test.

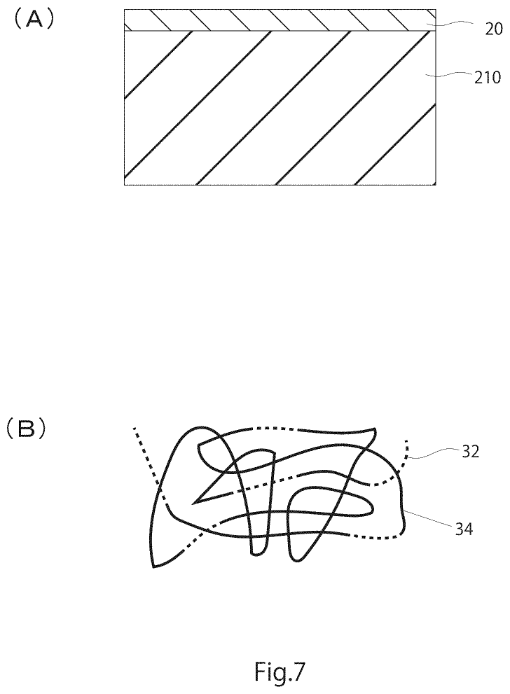

(Third Electronic functional member) With reference to FIG. 7, an electronic functional member according to a third embodiment of the invention (hereinafter referred to as a third electronic functional member) will be described. FIG. 7(A) is a schematic diagram for describing the third electronic functional member, and FIG. 7(B) is a schematic diagram illustrating an outline of a fiber network included in the third electronic functional member. FIG. 7(A) is a schematic cross-sectional view taken along a line similar to the line I-I of FIG. 1(A).

Similarly to the second electronic functional member, the third electronic functional member includes a fiber network 210 configured to include the first resin 32 and the second resin 34 which are water-soluble and different to one another in solubility to water after the thermocompression bonding, and the conductive member 20 formed on the fiber network 210. The third electronic functional member is configured to include a fiber containing both of the first resin 32 and the second resin 34 that are resins different in water solubility. As the first resin 32 and the second resin 34, for example, the resins similar to those of the second electronic functional member described above are usable.

Similarly to the second electronic functional member, also in the third electronic functional member, easily damaging the electrode with water during attaching to the skin was improved, and for example, the resistance against water was significantly improved without a damage regardless of attaching to the artificial skin and immersing in water for five minutes. The durability was significantly improved also in the wear resistance test without the wire disconnection until at least 1000 times.

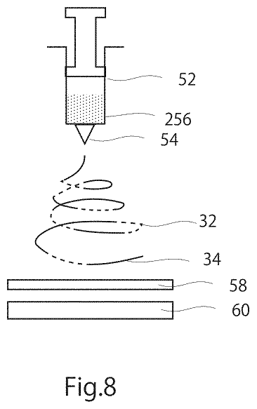

With reference to FIG. 8, a method for manufacturing the third electronic functional member will be described. In the third electronic functional member, as a solution, a solution 256 in which both of the first resin 32 and the second resin 34 are dissolved in a solvent is used. Consequently, a fiber containing both of the first resin 32 and the second resin 34 is formed through the needle 54. Because of the similarity to the method for manufacturing the first electronic functional member except that the solution in which the different two types of resins are dissolved is used, the description will be omitted.

While the example in which the different two types of resins are used and one resin changes to being poorly water-soluble by the heating and the pressure bonding is described as the configuration of the fiber network in the second electronic functional member and the third electronic functional member described above, the second electronic functional member and the third electronic functional member are not limited to this. It is only necessary to achieve the object of the present invention, and another resin other than the two types of the resins may be included.

The fiber network may be configured using one type of the resins. Even when the fiber network is configured using one type of the water-soluble resins, it becomes partially poorly water-soluble due to chemical or physical bonding force and/or cohesive force of its molecules in some cases. Also in this case, as a result, the fiber network that partially solved in water and partially remains is obtained.

The wear resistance in the case of using one type of resins is indicated in Table 2 below. The measurement was performed similarly to the friction and wear test in the first electronic functional member described above.

TABLE 2

| | the resin constituting the | the number of times until |
|---|---|---|
| | fiber network | the wire disconnection |
| example 10 | PVA derivative A (average | 10 |
| | viscosity: 50 mPa · s) | |
| example 11 | PVA derivative B (average | □1000 |
| | viscosity: 20 mPa · s) | |
| example 12 | PVA derivative C (average | □250 |
| | viscosity: 15 mPa · s) | |

Table 2 indicates examples in which PVA derivatives having the different viscosities were used as the resin constituting the fiber network. Examples 10 to 12 indicate the cases in which a PVA derivative A having the average viscosity of 50 mPa·s, a PVA derivative B having the average viscosity of 20 mPa·s, and a PVA derivative C having the average viscosity of 15 mPa·s were used, respectively. While what is called a kinematic viscosity is used as the viscosity for discrimination in Table 1, a viscosity coefficient is used as the viscosity for discrimination in Table 2. The viscosity here is also measured under the condition of 4% of a water solution at 20° C. according to Japanese Pharmaceutical Excipients (JPE).

The PVA derivative A is a poorly water-soluble resin having the high viscosity. In this case, the wear resistance is poor.

The PVA derivative C is a water-soluble resin having the low viscosity. In this case, the wear resistance is good or poor depending on the state when attaching to the artificial skin.

The PVA derivative B has the viscosity between those of the PVA derivative A and the PVA derivative C, and is less likely to be solved in water compared with the PVA derivative C while indicating the water-soluble property. Therefore, when immersed in water, some parts are dissolved and other parts remain Consequently, the wear resistance is excellent.

Note that the PVA derivative A has an initial resistance value of 110 Ω or more and the network rate a obtained by the formula (2) described above of less than 0.05, and the PVA derivative B has an initial resistance value of 34 to 45 Ω and the network rate of 0.1 or more.

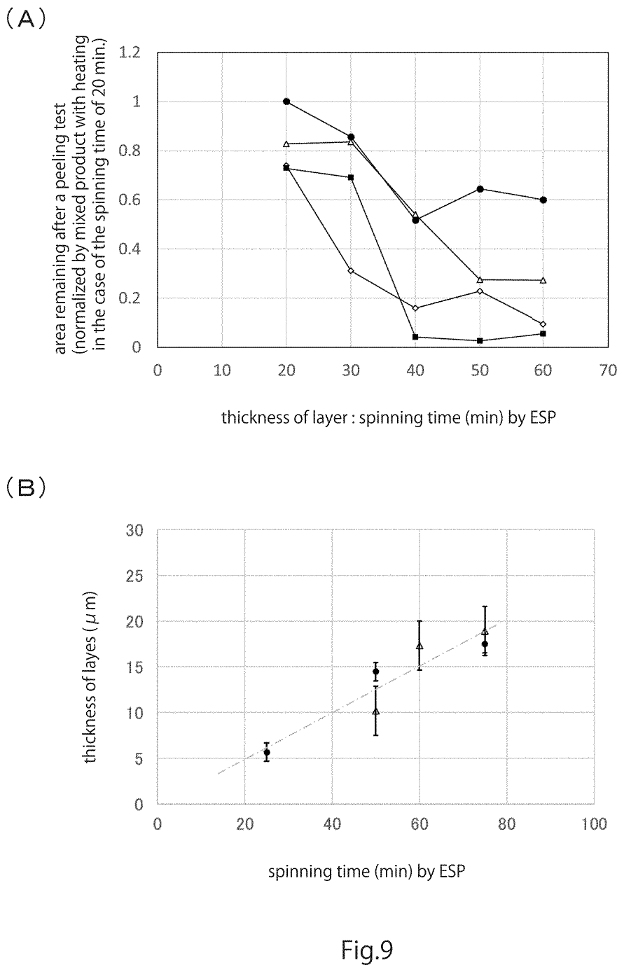

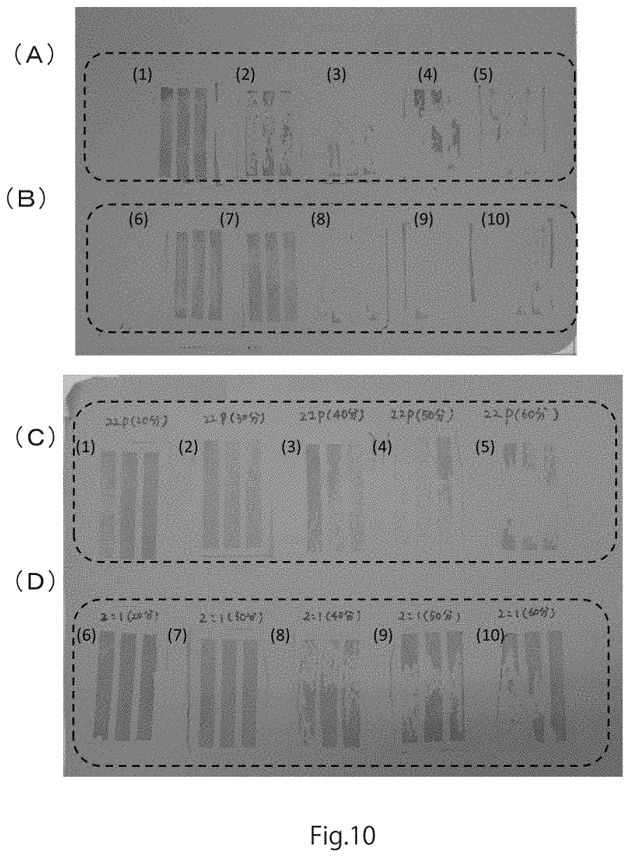

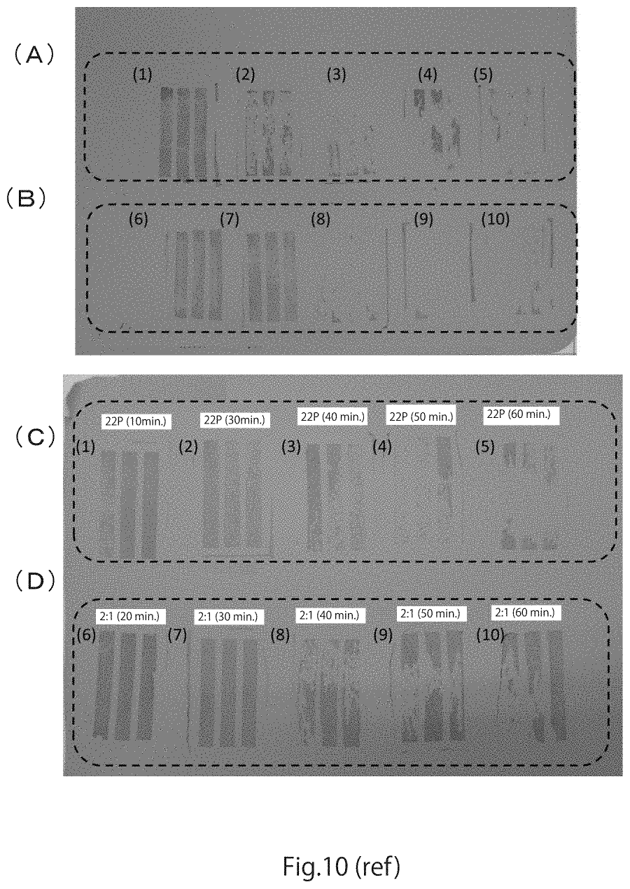

With reference to FIG. 9 and FIG. 10, an adhesion evaluation of the third electronic functional member will be described. FIGS. 9(A) and 9(B) are drawings illustrating the results of the adhesion evaluation.

In FIG. 9(A), the horizontal axis indicates a spinning time (minute) by an electrospinning apparatus, and the vertical axis indicates a normalized area remaining after a peeling test. In FIG. 9(A), an EG-22P single layer product without heating is indicated by outlined squares, an EG-22P single layer product heated at 130° C. is indicated by outlined triangles, a mixed product of EG-22P and EG-48P without heating is indicated by black squares, and a mixed product of EG-22P and EG-48P heated at 180° C. is indicated by black circles. Note that the vertical axis is normalized by the area after the peeling of the mixed product of EG-22P and EG-48P heated at 180° C. in the case of the spinning time of 20 minutes.

In FIG. 9(B), the horizontal axis indicates the spinning time (minute) by the electrospinning apparatus, and the vertical axis indicates a thickness (μm) of the nanomesh layer. Note that, in FIG. 9(B), for the thickness of the nanomesh layer, a result of measuring the thickness with a laser microscope is indicated by black circles, and the thickness measured from the cross-sectional surface of the nanomesh layer is indicated by outlined triangles.

FIG. 10 includes photographs illustrating the results of the adhesion evaluation. FIGS. 10(A) to 10(D) indicate the single layer product without heating, the mixed product without heating, the heated single layer product, and the heated mixed product, respectively. In the drawings, (1) and (6), (2) and (7), (3) and (8), (4) and (9), and (5) and (10) correspond to the spinning time of 20 minutes, 30 minutes, 40 minutes, 50 minutes, and 60 minutes, respectively.

Here, four types of the nanomeshes were attached to the artificial skin with water, and subsequently, the peeling test was conducted using a tape of JIS standard.

As a result of the adhesion evaluation, the single layer product without heating is high in adhesion in the case of the spinning time of 20 minutes, and the mixed product without heating, the heated single layer product, and the heated mixed product are high in adhesion in the case of the spinning time of 20 minutes to 30 minutes. That is, it is indicated that the thinner the thickness of the nanomesh layer is, the higher the adhesion is.

While it is indicated that the adhesion of the heated mixed product is high among the four types of the nanomeshes, especially, the adhesion is the highest in the heated mixed product with the spinning time of 20 minutes.

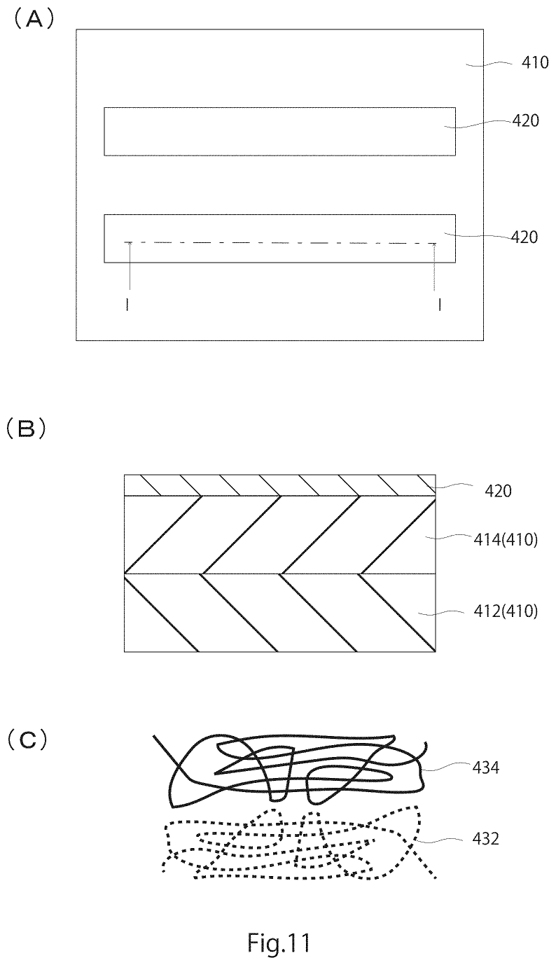

(Fourth Electronic functional member) With reference to FIG. 11, an electronic functional member according to a fourth embodiment of the invention (hereinafter referred to as a fourth electronic functional member) will be described. FIG. 11 includes schematic diagrams for describing the fourth electronic functional member. FIG. 11(A) is a schematic plan view, FIG. 11(B) is a schematic cross-sectional view taken along a line I-I of FIG. 11(A), and FIG. 11(C) is a schematic diagram illustrating a fiber network included in the fourth electronic functional member.

The fourth electronic functional member is configured to include a fiber network 410 and a conductive member 420 formed on the fiber network 410. The fiber network 410 is configured by stacking a first fiber network 412 and a second fiber network 414. The conductive member 420 is disposed on the second fiber network 414 of the fiber network 410.

While the first fiber network 412 and the second fiber network 414 are formed by any appropriate conventionally-known method, for example, similarly to the first electronic functional member, the first fiber network 412 and the second fiber network 414 are formed by injecting a resin composition by an electrospinning method.

The first fiber network 412 includes a first resin 432 solved in water when immersed in water. The first resin 432 is, for example, a water-soluble resin having a basic structure indicated by the formula (1) described above. As the first resin 432, EG-22P of GOHSENOL EG series by Mitsubishi Chemical Corporation is available.

The second fiber network 414 is configured to include a water-insoluble second resin 434 that is not solved in water and remains when immersed in water. As the water-insoluble second resin 434, for example, polyurethane is used.

The first fiber network 412 and the second fiber network 414 are stacked and bonded by any appropriate means, thereby obtaining the fiber network 410. As another example, by forming the first fiber network 412 by the electrospinning method and subsequently forming the second fiber network 414 by the electrospinning method, the fiber network 410 is obtained.

Since the second resin 434 constituting the second fiber network 414 is not dissolved when immersed in water, the second resin 434 almost entirely remains while the first resin 432 constituting the first fiber network 412 is entirely or partially solved in water when immersed in water for a predetermined time.

The conductive member 420 can be formed using, for example, an evaporation method, a sputtering method, a chemical vapor deposition method, an inkjet method, a screen-printing method, a gravure printing method, and a flexography method. While FIG. 11(A) illustrates an exemplary configuration of two strip shapes as the electrodes including the conductive members 20, an appropriate shape may be employed depending on the usage.

The shape of the electrode including the conductive member 420 can be changed by performing a patterning as necessary. As the patterning method, film formation via a mask is the easiest and preferred.

The material constituting the conductive member 420 only needs to have a conductive property. For example, a metal such as copper, gold, aluminum, silver, and zinc is usable. From the aspect of the conductive property, particularly, copper and silver are preferred. In a case of using for a living body and the like, for suppressing unwanted reactions, the use of stable gold is preferred.

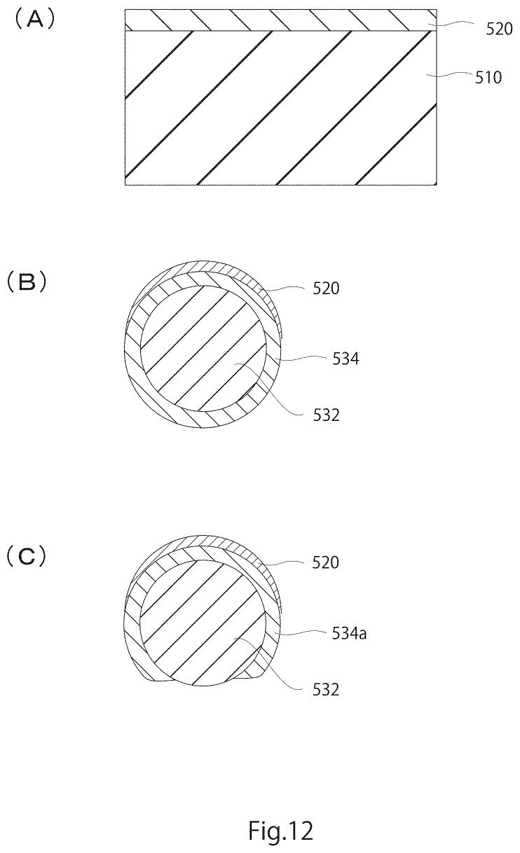

(Fifth Electronic functional member) With reference to FIG. 12, an electronic functional member according to a fifth embodiment of the invention (hereinafter referred to as a fifth electronic functional member) will be described. FIG. 12 includes schematic diagrams for describing the fifth electronic functional member. FIG. 12(A) is a schematic plan view, FIGS. 12(B) and 12(C) are schematic diagrams illustrating cross-sectional surfaces of a fiber included in the fifth electronic functional member.

The fifth electronic functional member is configured to include a fiber network 510 and a conductive member 520 formed on the fiber network 510. The fiber network 510 includes a water-soluble first resin 532 and a water-insoluble second resin 534. The first resin 532 is, similarly to the fourth electronic functional member, for example, a water-soluble resin having the basic structure indicated by the formula (1) described above. As the second resin 534, for example, polyparaxylene is used.

The fiber constituting the fiber network 510 is configured to include the first resin 532 and the second resin 534 that covers the whole or a part of a surface of the fiber including the first resin 532. The fiber positioned in the lower most layer of the fiber network 510 has a lower portion from which the first resin 532 is exposed (see FIG. 12(C)). In the fiber at positions other than the lower most layer of the fiber network 510, the second resin 534 covers the whole surface of the first resin 532 (see FIG. 12(B)).

Since the conductive member 520 can be configured similarly to the fourth electronic functional member, the overlapping explanation is omitted.

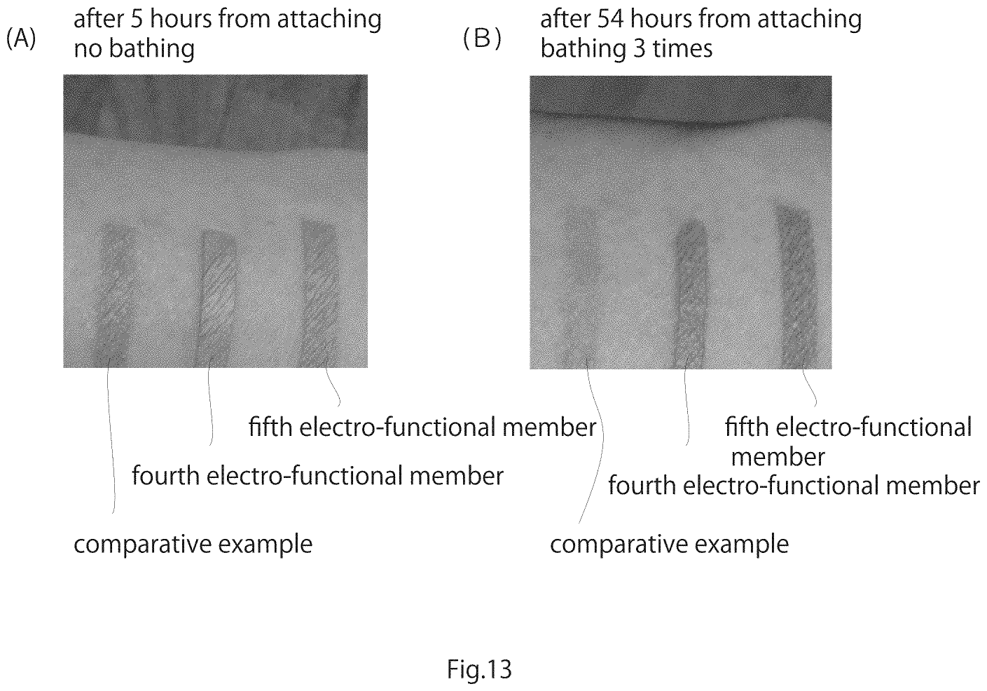

(Water Resistance Test) With reference to FIG. 13, the water resistance test of the fourth electronic functional member and the fifth electronic functional member will be described. FIG. 13 includes photographs illustrating the results of the water resistance test of the fourth electronic functional member and the fifth electronic functional member.

Nanomesh electrodes as the fourth electronic functional member and the fifth electronic functional member were attached to skins in inner sides of human forearms by exposing the nanomesh electrodes to water vapor. As a comparative example, an electronic functional member in which a fiber network was formed with only a water-soluble resin without using a water-insoluble resin and a conductive member was formed thereon was also attached to a skin in an inner side of a human forearm by exposing the electronic functional member to water vapor.

In FIG. 13(A) and FIG. 13(B), the comparative example, the fourth electronic functional member, and the fifth electronic functional member are illustrated from the left side. In the fourth functional member, the second fiber network of polyurethane (PU) is disposed on the first fiber network of EG-22P, and the conductive layer of gold (Au) is disposed on the second fiber network. In the fifth electronic functional member, polyparaxylene (parylene) is provided on the surface of the fiber of EG-22P, and the conductive layer of gold (Au) is disposed on the surface of the polyparaxylene. Meanwhile, in the comparative example, the conductive layer of gold (Au) is disposed on the fiber network of EG-22P.

FIG. 13(A) illustrates the state after five hours from the attaching, and FIG. 13(B) illustrates the state after 54 hours from the attaching. A bath was not taken until the elapse of five hours after the attaching, and then, the bath was taken three times until the elapse of 54 hours after the attaching.

In the comparative example, after the elapse of 54 hours from the attaching with the bathing at three times, there is almost no remaining. Meanwhile, it was confirmed that the fourth electronic functional member and the fifth electronic functional member were firmly attached to the skins even after the elapse of 54 hours from the attaching with the bathing at three times.

Thus, it was shown that the water resistance was improved in the fourth electronic functional member and the fifth electronic functional member compared with the conventional configuration indicated as the comparative example.

(Usage Example) The first to fifth electronic functional members (hereinafter also referred to as nanomesh electrodes) including the fiber networks of nanofiber (also referred to as nanomesh) are excellent in permeabilities of gas and water content. Therefore, the nanomesh electrodes can be attached to the living body surface for a long time.

Here, in the measurement of a living body signal, such as a measurement of an electrocardiogram and a skin resistance, a configuration of also attaching a measurement module to the living body surface is employed. In this case, the measurement module itself is often poor in air permeability, and the characteristics of the nanomesh electrode, excellent permeabilities of gas and water content, are not utilized in some cases.

Therefore, the inventors involved in this application have examined and have reached a biometric sensor excellent in air permeability as a whole even when the measurement module itself is poor in air permeability.

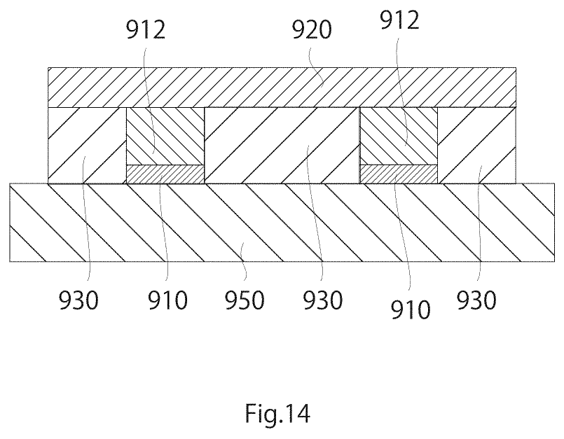

With reference to FIG. 14, the biometric sensor will be described. FIG. 14 is a schematic diagram for describing an exemplary configuration of the biometric sensor.

The biometric sensor is configured to include one or a plurality of nanomesh electrodes 910, a measurement module 920, air permeable electrodes 912 disposed between the measurement module 920 and the respective nanomesh electrodes 910, and an air permeable member 930. The nanomesh electrodes 910, the air permeable electrodes 912, and the air permeable member 930 are disposed in the same side with respect to the measurement module 920.

As the air permeable electrode 912, for example, a sponge electrode that has a conductive property, a porous structure, and an elasticity, and an electrode having a structure in which conductive threads are knitted to provide an air permeability and an elasticity are included. As the air permeable member 930, for example, a sponge that is made of a non-conductive resin material and has a porous structure and an elasticity is used.

To a living body 950 as a measurement target, the nanomesh electrodes 910 and the air permeable member 930 are attached to be contacted. Between the measurement module 920 and the living body 950, the nanomesh electrode 910 and the air permeable electrode 912, or the air permeable member 930 is disposed. Thus, since the members having the air permeability are disposed between the measurement module 920 and the living body 950, the biometric sensor is excellent in air permeability as a whole even when the measurement module 920 itself is poor in air permeability. Accordingly, the possibility of causing a skin inflammation due to the reduction of the air permeability of the skin surface can be reduced regardless of attaching to the living body surface for a long time, thus allowing reducing an itch and a rash of the attached measurement target.

### DESCRIPTION OF REFERENCE SIGNS

10, 110, 210, 410, 510 Fiber network 12, 412 First fiber network

14, 414 Second fiber network

20, 420, 520 Conductive member

30 Resin composition

32, 432, 532 First resin

34, 434, 534 Second resin

52, 152, 153 Syringe

54, 154, 155 Needle

56, 156, 157, 256 Solution

58 Support body

60 Conductive sheet

1. An electro-functional member comprising:
a fiber network configured to include a resin, the fiber network being partially solved in water and partially remaining when immersed in water; and
a conductive member formed on the fiber network.

2. The electro-functional member according to claim 1, wherein
the resin is a polyvinyl alcohol derivative.

3. The electro-functional member according to claim 1 or 2, wherein
the fiber network includes a first resin and a second resin as the resin, and the first resin and the second resin are mutually different in solubility to water.

4. The electro-functional member according to claim 3, wherein
the fiber network is configured by stacking:
a first fiber network including a fiber containing the first resin; and
a second fiber network including a fiber containing the second resin.

5. The electro-functional member according to claim 3, wherein
the fiber network includes a fiber containing the first resin and a fiber containing the second resin

6. The electro-functional member according to claim 3, wherein
the fiber network includes a fiber containing the first resin and the second resin.

7. The electro-functional member according to claim 1, wherein
the fiber network is configured by stacking:
a first fiber network including a fiber containing the first resin; and
a second fiber network including a fiber containing the second resin,
the first resin is a water-soluble resin, and
the second resin is a water-insoluble resin.

8. The electro-functional member according to claim 7, wherein
the first resin is a polyvinyl alcohol derivative, and
the second resin is polyurethane.

9. The electro-functional member according to claim 1, wherein
the fiber network includes a fiber containing the first resin and a fiber containing the second resin,
the first resin is a water-soluble resin,
the second resin is a water-insoluble resin, and
the second resin is disposed to cover an entire or a part of a surface of the fiber of the first resin.

10. The electro-functional member according to claim 9, wherein
the first resin is a polyvinyl alcohol derivative, and
the second resin is polyparaxylene.

11. The electro-functional member according to any one of claims 1 to 10, wherein
an occupancy of the fiber in the fiber network is 20% to 90%.

12. The electro-functional member according to any one of claims 1 to 11, wherein
the occupancy of the fiber in the fiber network is 30% to 70%.

13. A method for manufacturing an electro-functional member, comprising:
a step of forming a first fiber network that includes a fiber containing a water-soluble first resin;
a step of forming a second fiber network that includes a fiber containing a second resin that is water-soluble and changes to being poorly water-soluble after heating and pressure bonding;
a step of changing the second resin to being poorly water-soluble by heating and pressure bonding the second fiber network; and
a step of stacking the first fiber network, the second fiber network, and a conductive member.

14. A method for manufacturing an electro-functional member, comprising:
a step of forming a fiber network that includes a fiber containing a first resin and a fiber containing a second resin, the first resin being water-soluble and remaining water-soluble after heating and pressure bonding, and the second resin being water-soluble and changing to being poorly water-soluble after the heating and the pressure bonding;
a step of heating and pressure bonding the fiber network; and
a step of disposing a conductive member on the fiber network.

15. A method for manufacturing an electro-functional member, comprising:
a step of forming a fiber network that includes a fiber containing a first resin and a second resin, the first resin being water-soluble and remaining water-soluble after heating and pressure bonding, and the second resin being water-soluble and changing to being poorly water-soluble after the heating and the pressure bonding; and
a step of heating and pressure bonding the fiber network cork and further disposing a conductive member on the fiber network.

16. A method for manufacturing an electro-functional member, comprising:
a step of forming a first fiber network that includes a fiber containing a watt soluble first resin
a step of forming a second fiber network that includes a fiber containing a water-insoluble second resin; and
a step of stacking the first fiber network, the second fiber network, and a conductive member.

17. A method for manufacturing an electro-functional member, comprising:
a step of forming a fiber network by disposing a water-insoluble second resin on a surface of a fiber that includes a water-soluble first resin; and
a step of stacking a conductive member on the fiber network.

18. A biometric sensor comprising.
one or a plurality of nanomesh electrodes;
a measurement module;
an air permeable electrode disposed between the measurement module and the nanomesh electrode; and
an air permeable member, wherein
the nanomesh electrode, the air permeable electrode, and the air permeable member are disposed in an identical side with respect to the measurement module,
the nanomesh electrode and the air permeable member are attached to contact a measurement target, and
the nanomesh electrode is the electro-functional member according to any one of claims 1 to 12.
